(19) United States
(12) Patent Application Publication
(10) Pub. No.: US 2009/0076261 A1
(43) Pub. Date: Mar. 19, 2009

(54) Novel process for preparing sucrose-6-esters

(75) Inventor: Shaojun Xu, Hangzhou (CN)

Correspondence Address: BUCHANAN, INGERSOLL & ROONEY PC, POST OFFICE BOX 1404, ALEXANDRIA, VA, 22313-1404, US

(73) Assignee: POLYMED THERAPEUTICS, INC., Houston, TX (US)

(21) Appl. No.: 11/898,652

(22) Filed: Sep. 13, 2007

(51) Int. Cl. C07H 1/00

(52) U.S. Cl. 536/127; 536/124

(57) ABSTRACT

A process for preparing a sucrose-6-ester, a key intermediate to sucralose. The process contains (a) reacting sucrose with a di(hydrocarbyl)tin oxide or a 1,3-diacyloxy-1,1,3,3-tetra(hydrocarbyl)distannoxane in the presence of a secondary alcohol and an organic polar aprotic solvent, to prepare a mixture comprising 1,3-di-(6-O-sucrose)-1,1,3,3-tetra(hydrocarbyl)distannoxane and the secondary alcohol; and (b) adding an acylating agent to the mixture, thereby acylating the 1,3-di-(6-O-sucrose)-1,1,3,3-tetra(hydrocarbyl)distannoxane to prepare a sucrose-6-ester.

### BACKGROUND OF THE INVENTION

1. Field of the Invention

The present invention relates to processes for preparing sucrose-6-esters. In particular, a sucrose-6-ester can be prepared by reacting sucrose with a di(hydrocarbyl)tin oxide or a 1,3-diacyloxy-1,1,3,3-tetra(hydrocarbyl)distannoxane in the presence of a secondary alcohol and an organic polar aprotic solvent, followed by acylation.

2. Description of the Related Art

Sucralose is a high intensity artificial sweetener, which has been used as a food sweetener in many countries since its discovery in the 1970s. The chemical structure of sucralose is represented by the following formula (I):

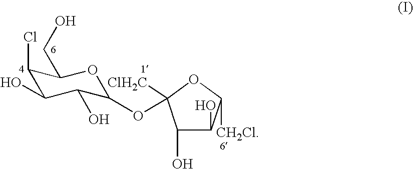

There are a number of processes for the preparation of sucralose, many of which involve selective chlorination of a sucrose-6-ester as a key step, followed by deacylation.

The sucrose molecule contains three primary hydroxyl groups and five secondary hydroxyl groups. Therefore, it can be technically challenging to prepare a sucrose-6-ester as a major product from sucrose in a one-step process. Several methods for the preparation of sucrose-6-esters are disclosed, for example, in U.S. Pat. Nos. 4,950,746, 5,023,329 and 5,089,608, where sucrose is reacted with a di(hydrocarbyl)tin oxide or derivative thereof to first prepare a sucrose organotin compound, which, in turn, is acylated with an acylating agent to prepare a sucrose-6-ester.

Specifically, in U.S. Pat. No. 4,950,746, sucrose, dibutyltin oxide and methanol are boiled under reflux to afford 1,3-di-(O-sucrose)-1,1,3,3-tetrabutyldistannoxane. At this stage, the methanol and water are stripped off to obtain a white solid. The white solid is then dissolved in N,N-dimethylformamide (DMF) and reacted with an acylating agent, in particular, benzoic anhydride, to form sucrose-6-benzoate. This process involves recovery of the intermediate compound, and thus is procedurally complicated and not cost-effective for industrial scale productions.

In U.S. Pat. No. 5,023,329, sucrose and di(hydrocarbyl)tin oxide are reacted under reflux, with removal of water, in an inert mixed organic solvent system such as a solvent system containing DMF and cyclohexane. The resulting sucrose organotin compound is then reacted with an acylating agent, in particular, a carboxylic acid anhydride, to afford a sucrose-6-ester.

In U.S. Pat. No. 5,089,608, a di(hydrocarbyl)tin oxide is reacted with a dihydric alcohol, for example, ethylene glycol, in an inert organic vehicle such as hydrocarbons having boiling points between about 80° C. to about 145° C., to form a cyclic adduct of the di(hydrocarbyl)tin oxide and the dihydric alcohol. After removal of the solvent and excessive dihydric alcohol under high vacuum, the resulting solid is dissolved in DMF and reacted with sucrose, followed by the treatment with an acylating agent. In this process, high vacuum drying is required due to the relatively high boiling points of dihydric alcohols and often is inconvenient for industrial scale productions. In addition, during the acylation reaction, it is necessary to employ two molar equivalents of the acylating agent, even though only one equivalent is used to produce the desired compound. This is because when the cycloadduct, for example, 2,2-di(hydrocarbyl)-1,3-dioxa-2-stannolane, is reacted with sucrose, an intermediate compound, i.e., HOCH₂CH₂OSn(hydrocarbyl)-O-Suc is produced. This intermediate compound contains a primary hydroxyl group which can consume one molar equivalent of the acylating agent.

There have been studies on selective acylation of a single hydroxy group in diols and polyalcohols in the presence of an organotin catalyst. For example, in Regioselective Manipulation of Hydroxyl Groups via Organotin Derivatives by David et al., Tetrahedron, vol. 41(4), pages 643-63 (1985), the authors report that the reactions of tin compounds with hydroxyl group-containing compounds produce stannoxyl compounds, which can then be alkylated or acylated to produce ethers or esters. Particularly, the reaction of bis(tributyltin)oxide with various carbohydrates including sucrose, followed by acylation, produces a mixture of esters of varying degrees of substitution. The use of dibutyltin oxide in a reaction with a hydrocarbon is also described in this article.

In addition, Morcuende et al. describe selective mono-acylation of diols and polyols with acyl chloride under microwave and in the presence of dibutyltin oxide catalyst and an organic base. See, Rapid Formation of Dibutylstannylene Acetals from Polyhydroxylated Compounds under Microwave Heating. Application to the Regioselective Protection of Polyols and to a Catalytic Tin-Mediated Benzoylation, Synlett, 89-91 (1994), and Microwave Accelerated Organic Transformations: Dibutylstannylene Acetal Mediated Selective Acylation of Polyols and Amino Alcohol Using Catalytic Amounts of Dibutyltin Oxide. Influence of the Solvent and the Power Output on the Selectivity, Synlett, 455-58 (1995). Further, Boons et al. describe in Selective Acylation and Alkylation Reactions of Diols Using Dibutyltin Dimethoxide, Synlett, 913-15 (1993), selective protection of primary hydroxyl with acyl chloride using organotin acetal in the presence of an organic base catalyst. See, also, Dibutyltin Oxide Catalyzed Selective Sulfonylation of α-Chelatable Primary Alcohols by Martinelli et al., Organic Letters, vol. 1(3), 447-50 (1999); and Chemo-and Stereo-Selective Monobenzoylation of 1,2-Diols Catalyzed by Organotin Compounds by Iwasaki et al., J. Org. Chem., 65, 996-1002 (2000).

Further, in Diol-Tin Ketal as Effective Catalyst in the Tin Mediated Benzoylation of Polyols by Fasoli et al., Journal of Molecular Catalysis A: Chemical, 244, 41-45 (2006), the authors mention that dibutyltin diisopropoxide can catalyze selective mono-acylation of diol compounds in isopropanol solvent. In particular, the authors report no significant acylation between an acid chloride and the solvent when the molar ratio of diol and benzoic chloride is 1:1.1.

### SUMMARY OF THE INVENTION

The present application provides a process for preparing a sucrose-6-ester, a key intermediate to sucralose, comprising:

(a) reacting sucrose with a di(hydrocarbyl)tin oxide or a 1,3-diacyloxy-1,1,3,3-tetra(hydrocarbyl)distannoxane in the presence of a secondary alcohol and an organic polar aprotic solvent, to prepare a mixture comprising 1,3-di-(6-O-sucrose)-1,1,3,3-tetra(hydrocarbyl)distannoxane and the secondary alcohol; and

(b) adding an acylating agent to the mixture, thereby acylating the 1,3-di-(6-O-sucrose)-1,1,3,3-tetra(hydrocarbyl)distannoxane to prepare a sucrose-6-ester.

As described herein, there is no need to replace the reaction medium for the formation of intermediate 1,3-di-(6-O-sucrose)-1,1,3,3-tetra(hydrocarbyl)distannoxanes and of final product sucrose-6-esters, thereby providing a simplified and cost-effective one-pot production of sucrose-6-esters from sucrose.

### DETAILED DESCRIPTION OF THE PREFERRED EMBODIMENTS

As described herein, sucrose can be first reacted (or condensed) with a di(hydrocarbyl)tin oxide or a 1,3-diacyloxy-1,1,3,3-tetra(hydrocarbyl)distannoxane (“distannoxane diester” or “DSDE”) in the presence of a secondary alcohol and an organic polar aprotic solvent, to prepare a mixture comprising 1,3-di-(6-O-sucrose)-1,1,3,3-tetra(hydrocarbyl)distannoxane (“DBSS”) and the secondary alcohol, as shown below:

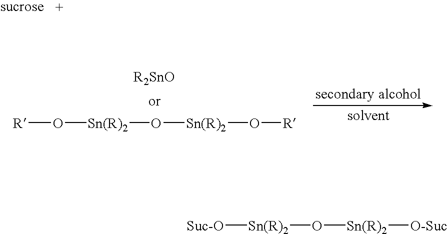

The term “hydrocarbyl,” as used herein, denotes an alkyl, cycloalkyl, aryl or aralkyl group.

The hydrocarbyl groups in the di(hydrocarbyl)tin oxide are the same or different, and each can preferably represent an alkyl group having from 1 to 12, more preferably, from 2 to 10, and most preferably, from 4 to 8, carbon atoms. Preferred examples of di(hydrocarbyl)tin oxides may include, but are not limited to, dibutyltin oxide and dioctyltin oxide. More preferably, the di(hydrocarbyl)tin oxide comprises dibutyltin oxide.

The 1,3-diacyloxy-1,1,3,3-tetra(hydrocarbyl)distannoxane can be represented by the formula:

R′CO—O—Sn(R)₂—O—Sn(R)₂—O—COR′

wherein each R′ individually represents alkyl, cycloalkyl, aryl or aralkyl, and each R individually represents a hydrocarbyl group, such as alkyl, cycloalkyl, aryl and aralkyl.

Preferably, each R′ individually represents a C₁-C₈ alkyl group or phenyl. Examples of preferred C₁-C₈ alkyl groups may include, but are not limited to, methyl, ethyl, n-propyl, n-butyl, n-pentyl and n-hexyl.

In a preferred embodiment, each R individually represents a C₁-C₈ alkyl group, and more preferably, a butyl group.

Examples of preferred 1,3-diacyloxy-1,1,3,3-tetra(hydrocarbyl)distannoxanes may include, but are not limited to, 1,3-diacetoxy-1,1,3,3-tetrabutyldistannoxane (“distannoxane diacetate” or “DSDA”) and 1,3-dibenzoyloxy-1,1,3,3-tetrabutyldistannoxane (“distannoxane dibenzoate” or “DSDB”). More preferably, the 1,3-diacyloxy-1,1,3,3-tetra(hydrocarbyl)distannoxane comprises 1,3-diacetoxy-1,1,3,3-tetrabutyldistannoxane.

As described herein, the secondary alcohol is capable of forming an azeotrope with water. In one embodiment, the secondary alcohol contains only one hydroxyl group in its molecule. Examples of suitable secondary alcohols may include, but are not limited to, isopropanol, 2-butanol, 2-pentanol and 3-pentanol. Preferably, the secondary alcohol comprises isopropanol.

Examples of suitable organic polar aprotic solvents may include, but are not limited to, N,N-dimethylformamide (“DMF”), N-methyl-2-pyrrolidone (“NMP”), dimethylsulfoxide (“DMSO”), N,N-dimethylacetamide (“DMA”) and hexamethylphosphoramide (“HMPA”). These solvents may be used individually or in combination thereof. Preferably, the solvent comprises N,N-dimethylformamide.

As described herein, sucrose and a di(hydrocarbyl)tin oxide or a 1,3-diacyloxy-1,1,3,3-tetra(hydrocarbyl)distannoxane can be mixed with a secondary alcohol and an organic polar aprotic solvent, to form the corresponding 1,3-di-(6-O-sucrose)-1,1,3,3-tetra(hydrocarbyl)distannoxane represented by the formula:

Suc-O—Sn(R)₂—O—Sn(R)₂—O-Suc

wherein R has the same meaning as defined above.

When a di(hydrocarbyl)tin oxide is used in the condensation reaction, the molar ratio of sucrose: di(hydrocarbyl)tin oxide preferably ranges from about 1:1 to about 1:1.5, and more preferably, from about 1:1.05 to about 1:1.2. The term “about,” as used herein, denotes a range of ±20%, preferably ±10%, more preferably ±5%, further preferably, ±2%, and most preferably ±1%.

When a 1,3-diacyloxy-1,1,3,3-tetra(hydrocarbyl)distannoxane is used in the condensation reaction, the molar ratio of sucrose: 1,3-diacyloxy-1,1,3,3-tetra(hydrocarbyl)distannoxane preferably ranges from about 1:0.8 to about 1:1.5, and more preferably, from about 1:1 to about 1:1.2.

The amounts of the secondary alcohol and the organic polar aprotic solvent used in the condensation reaction are not particularly limited. In a preferred embodiment, the secondary alcohol can be used in an amount (by volume, milliliter) of about 1 to 12 times, and more preferably, about 4 to 10 times, the weight of sucrose (by weight, gram). In addition, the organic polar aprotic solvent can preferably be used in an amount (by volume, milliliter) of about 1 to 6 times, and more preferably, about 3 to 5 times, the weight of sucrose (by weight, gram).

As described herein, the mixture containing sucrose, di(hydrocarbyl)tin oxide or 1,3-diacyloxy-1,1,3,3-tetra(hydrocarbyl)distannoxane, secondary alcohol and organic polar aprotic solvent can be heated to reflux, preferably under an inert atmosphere, such as a nitrogen or argon atmosphere. The condensation reaction can be carried out at a temperature and for a time period sufficient to form the desired 1,3-di(6-O-sucrose)-1,1,3,3-tetra(hydrocarbyl)distannoxane. In one embodiment, the reaction temperature ranges from about 80° C. to about 140° C. and preferably, from about 90° C. to about 110° C.

To further drive the condensation reaction to completion, it is desirable to remove water produced by the condensation reaction during the reaction. Water can be removed from the reaction mixture by distillation as an azeotrope with the secondary alcohol. Typically, only a portion of the originally added amount of the secondary alcohol is needed to form an azeotrope with, and thereby remove, substantially all the water generated during the reaction. In one embodiment, at the end of the condensation reaction, about 10% to about 90%, or about 20% to about 80%, or about 30% to about 70%, of the originally added amount of the secondary alcohol remains in the reaction mixture. More preferably, about 40% to about 60% of the original amount of the secondary alcohol remains in the reaction mixture.

As described herein, no significant interference of secondary alcohols in the acylation reaction was observed. Thus, when the condensation reaction is complete, it is not necessary to remove substantially all the secondary alcohol from the reaction system at this stage. That is, the mixture contains not only the resulting 1,3-di-(6-O-sucrose)-1,1,3,3-tetra(hydrocarbyl)distannoxane, it can also contain the remaining secondary alcohol.

This mixture can be used directly for the acylation reaction, which makes it possible to prepare a sucrose-6-ester from sucrose in a one-pot process. The acylation reaction can typically be carried out by cooling the mixture to about 10° C. or below, which can then be treated with an acylating agent, such as a carboxylic acid anhydride, an acyl chloride and the like. Preferably, the acylating agent comprises a carboxylic acid anhydride, such as a substituted or unsubstituted, linear or branched C₁-C₄ alkanecarboxylic acid anhydride or a substituted or unsubstituted phenoic acid anhydride. The substituents in the substituted linear or branched C₁-C₄ alkanecarboxylic acid anhydride and the substituted phenoic acid anhydride may include, but are not limited to, C₁-C₄ alkyls and C₆-C₁₀ aryls.

When an acyl chloride is used as the acylating agent, a basic compound, such as triethylamine, can also be used additionally.

Examples of suitable carboxylic acid anhydrides may include, but are not limited to, acetic anhydride, benzoic anhydride, 4-methylbenzoic anhydride and 2-phenylacetic anhydride. Preferably, the carboxylic acid anhydride comprises acetic anhydride or benzoic anhydride. The amount of the acylating agent, in particular, the carboxylic acid anhydride, can preferably be about 1 to 1.5 times, and more preferably, 1.1 to 1.2 times by mole, the amount of sucrose.

The acylation reaction can be carried out at a temperature and for a time period sufficient to form the desired sucrose-6-esters. Typically, the resulting mixture of 1,3-di-(6-O-sucrose)-1,1,3,3-tetra(hydrocarbyl)distannoxane and acylating agent can be allowed to react at the initial temperature for about 1 hour and then warm naturally to room temperature to react until the acylation reaction is substantially complete. In one embodiment, the acylation reaction can be carried out at a temperature ranging from about −10° C. to about 45° C., and preferably, from about 0° C. to about 35° C. The reaction time can range from about 1 hour to about 15 hours, and more preferably, from about 3 hours to about 6 hours. In a preferred embodiment, the acylation reaction can be carried out under an inert atmosphere, such as a nitrogen or argon atmosphere.

After the acylation reaction is substantially complete, the resulting reaction mixture can be worked up. Typically, the organic tin-containing by-products can be extracted with an alkane solvent, thereby separated from the reaction mixture. Examples of suitable alkane solvents may include, but are not limited to, cyclohexane, n-hexane, n-heptane and the like. These alkane solvents may be used individually or in combination thereof. If desired, a small amount of water may be added to the reaction mixture prior to extraction with an alkane solvent.

The reaction mixture after extraction can then be concentrated to remove the remaining solvents, in particular, the secondary alcohol and the organic polar aprotic solvent, to afford a sucrose-6-ester. The resulting sucrose-6-ester can be used directly, or if desired, after further purification, in a process for preparing sucralose.

In the processes described herein, it is not necessary to remove the secondary alcohol after the formation of intermediate 1,3-di-(6-O-sucrose)-1,1,3,3-tetra(hydrocarbyl)distannoxanes. Furthermore, the solvent system used in the formation of 1,3-di-(6-O-sucrose)-1,1,3,3-tetra(hydrocarbyl)distannoxanes, which contains an organic polar aprotic solvent, can also be used as the medium in the acylation reaction, and thus there is no need to replace the reaction medium. In other words, there are fewer steps involved in these processes than those described in the art, which can greatly simplify the production operations. Moreover, the processes as described herein can provide economic advantages by utilizing the same solvent system in the formation of 1,3-di-(6-O-sucrose)-1,1,3,3-tetra(hydrocarbyl)distannoxanes and final product sucrose-6-esters. Further, these processes make it possible to prepare a sucrose-6-ester from sucrose in a one-pot process.

The present invention is further illustrated by the following specific examples but is not limited hereto.

### EXAMPLES

Unless specified, all the commercially available materials were used herein without further purification. All the measurements including weight and temperature were uncorrected.

### Example 1

### Preparation of 1,3-di-(6-O-sucrose)-1,1,3,3-tetrabutyl-distannoxane

A 250 mL three-neck round-bottom flask, equipped with a magnetic stir, a reflux condenser and a nitrogen gas inlet, was charged with dibutyltin oxide (25 g) and isopropanol (200 mL). The mixture was heated to reflux and stirred for 1 hour. Thereafter, an oil-water separator was placed between the reflux condenser and the flask. The reaction mixture was continued to reflux until about 100 mL of isopropanol and water was separated.

To the reminder mixture was added sucrose (25 g) and DMF (100 mL). The resulting mixture was refluxed for about 4.5 hours to separate additional 20 mL of isopropanol and water. At this stage, the reaction mixture became clear. The resulting clear mixture was cooled to room temperature to afford a 192.8 g solution containing 1,3-di-(6-O-sucrose)-1,1,3,3-tetrabutyl-distannoxane.

### Example 2

### Preparation of sucrose 6-acetate

A 50 mL single-neck round-bottom flask was charged with 20 g of the solution obtained in Example 1. The solution was cooled with an ice bath while stirring and under a nitrogen atmosphere. To this cooled solution was added acetic anhydride (1.2 mL). The resulting mixture was allowed to warm naturally to about 10° C. in about 1 hour. Thin layer chromatography (TLC) analysis by using CHCl₃:MeOH:H₂O (15:10:1) as an eluent showed that about 15% of sucrose remained unreacted. The reaction mixture was warmed to room temperature and stirred overnight. TLC analysis indicated that sucrose was consumed completely.

### Example 3

### Preparation of sucrose 6-benzoate

A 50 mL single-neck round-bottom flask was charged with 26 g of the solution obtained in Example 1. The solution was cooled with an ice bath while stirring and under a nitrogen atmosphere. To this cooled solution were added benzoic chloride (1.37 mL) and triethylamine (1.65 mL). The resulting mixture was allowed to warm naturally to about 10° C. in about 1 hour. Thin layer chromatography (TLC) analysis by using CHCl₃:MeOH:H₂O (15:10:1) as an eluent showed that about 75% of sucrose remained unreacted and that about 25% was converted to sucrose-6-benzoate. The reaction mixture was warmed to room temperature and stirred overnight. TLC analysis indicated that still about 60% of sucrose remained unreacted and that about 40% was converted to sucrose-6-benzoate.

### Example 4

### Preparation of sucrose 6-acetate

A 50 mL single-neck round-bottom flask was charged with 30.5 g of the solution obtained in Example 1. The solution was cooled with an ice bath while stirring and under a nitrogen atmosphere. To this cooled solution was added acetic anhydride (1.4 mL). The resulting mixture was allowed to warm naturally to about 15° C. in about 1 hour. Thin layer chromatography (TLC) analysis by using CHCl₃:MeOH:H₂O (15:10:1) as an eluent showed that about 20% of sucrose remained unreacted. The reaction mixture was warmed to room temperature and stirred for 4.5 hours.

The reaction mixture was then extracted with cyclohexane (30 mL×2). The remaining mixture after extraction was concentrated under a reduced pressure to remove the isopropanol and DMF to afford a sucrose 6-acetate syrup. High performance liquid chromatograph (HPLC) analysis indicated that the syrup consists of 82.3 wt. % of sucrose 6-acetate, 5.1 wt. % of sucrose, 10.2 wt. % of sucrose 4-acetate and 2.4 wt. % of other impurities. This syrup can be used directly, or after purification, if desired, in a process for preparing sucralose.

### Example 5

### Preparation of sucrose 6-acetate

A 100 mL three-neck round-bottom flask, equipped with a magnetic stir, a reflux condenser and a nitrogen gas inlet, was charged with dibutyltin oxide (5 g), sucrose (5.7 g), isopropanol (50 mL) and DMF (24 mL), the reaction mixture was heated to reflux and stirred for 1 hour. Then, an oil-water separator was placed between the reflux condenser and the flask.

The reaction mixture was continued to reflux at 100° C. for 4 hours until about 20 mL of isopropanol and water was separated.

Under a nitrogen atmosphere, the reaction mixture was cooled to 10° C. To this mixture was added acetic anhydride (2 mL). The resulting mixture was stirred at 17° C. for 1 hour, and then 27° C. for 2 hours. TLC analysis indicated about 20% of sucrose remained unreacted. The reaction mixture was stirred at room temperature for 3 hours.

The reaction mixture was then extracted with cyclohexane (50 mL×2). The remaining mixture after extraction was concentrated under a reduced pressure to remove the isopropanol and DMF to afford a syrup. HPLC analysis indicated that the syrup consists of 84 wt. % of sucrose 6-acetate, 3.5 wt. % of sucrose, 9.9 wt. % of sucrose 4-acetate and 2.6 wt. % of other impurities.

From the foregoing description and illustration of this invention it is apparent that various modifications may be made to produce similar results. It is the desire of the applicants not to be bound by the description of this invention as contained in the specification, but to be bound only by the claims as appended hereto.

All of the above-mentioned references are herein incorporated by reference in their entirety to the same extent as if each individual reference was specifically and individually indicated to be incorporated herein by reference in its entirety.

What is claimed is:

1. A process for preparing a sucrose-6-ester, comprising:
(a) reacting sucrose with a di(hydrocarbyl)tin oxide or a 1,3-diacyloxy-1,1,3,3-tetra(hydrocarbyl)distannoxane in the presence of a secondary alcohol and an organic polar aprotic solvent, to prepare a mixture comprising 1,3-di-(6-O-sucrose)-1,1,3,3-tetra(hydrocarbyl)distannoxane and the secondary alcohol; and
(b) adding an acylating agent to the mixture, thereby acylating the 1,3-di-(6-O-sucrose)-1,1,3,3-tetra(hydrocarbyl)distannoxane to prepare a sucrose-6-ester.

2. The process of claim 1, further comprising:
(c) recovering the sucrose-6-ester.

3. The process of claim 1, wherein the di(hydrocarbyl)tin oxide comprises dibutyltin oxide or dioctyltin oxide.

4. The process of claim 1, wherein the 1,3-diacyloxy-1,1,3,3-tetra(hydrocarbyl)distannoxane comprises 1,3-diacetoxy-1,1,3,3-tetrabutyldistannoxane or 1,3-dibenzoyloxy-1,1,3,3-tetrabutyldistannoxane.

5. The process of claim 4, wherein the 1,3-diacyloxy-1,1,3,3-tetra(hydrocarbyl)distannoxane comprises 1,3-diacetoxy-1,1,3,3-tetrabutyldistannoxane.

6. The process of claim 1, wherein the organic polar aprotic solvent comprises N,N-dimethylformamide, N-methyl-2-pyrrolidone, dimethylsulfoxide, N,N-dimethylacetamide, hexamethylphosphoramide, or a mixture thereof.

7. The process of claim 6, wherein the organic polar aprotic solvent comprises N,N-dimethylformamide.

8. The process of claim 1, wherein the secondary alcohol comprises isopropanol, 2-butanol, 2-pentanol or 3-pentanol.

9. The process of claim 8, wherein the secondary alcohol comprises isopropanol.

10. The process of claim 1, wherein the acylating agent comprises a carboxylic acid anhydride.

11. The process of claim 10, wherein the carboxylic acid anhydride comprises a substituted or unsubstituted, linear or branched C₁-C₄ alkanecarboxylic acid anhydride or a substituted or unsubstituted phenoic acid anhydride.

12. The process of claim 11, wherein the carboxylic acid anhydride comprises acetic anhydride or benzoic anhydride.

13. The process of claim 1, wherein the sucrose: dialkyltin oxide molar ratio ranges from about 1:1 to about 1:1.5.

14. The process of claim 1, wherein the sucrose: acylating agent molar ratio ranges from about 1:1 to about 1:1.5.

15. The process of claim 1, wherein the reacting (a) is carried out by heating to reflux and removal of water.

16. The process of claim 1, wherein the reacting (a), the adding (b) or both are conducted under an inert atmosphere.

17. The process of claim 1, wherein the adding (b) is conducted at a temperature ranging from about −10° C. to about 45° C.

18. The process of claim 2, wherein the recovering (c) comprises:
(i) removing residue organotin compounds from the mixture obtained in (b) by extraction with an alkane solvent, and
(ii) removing the solvents from the mixture obtained in (i).

19. The process of claim 18, wherein the alkane solvent comprises cyclohexane, n-hexane, n-heptane or a combination thereof.
